(19) United States
(12) Patent Application Publication
(10) Pub. No.: US 2020/0253701 A1
(43) Pub. Date: Aug. 13, 2020

(54) SUCTION APPARATUS AND SALIVA EJECTOR

(71) Applicant: Daniel Tevrizian, San Marcos, CA (US)

(72) Inventor: Daniel Tevrizian, San Marcos, CA (US)

(21) Appl. No.: 16/272,109

(22) Filed: Feb. 11, 2019

(51) Int. Cl. A61C 17/08; A61C 17/06; A61D 5/00; A61C 17/14; A61L 29/00

(52) U.S. Cl. CPC A61C 17/08; A61C 17/096; A61L 29/005; A61C 17/14; A61D 5/00

(57) ABSTRACT

The present invention provides an apparatus adapted for ejection of material, e.g., saliva, from the mouth of a patient during a dental procedure. The apparatus has at least two distal openings such that material may be ejected simultaneously from at least two locations within the patient's mouth. The invention provides a suction apparatus having a proximal tube, which may couple with a standard waste container apparatus having a suction source.

### FIELD OF THE INVENTION

The invention generally relates to apparatus adapted for ejection of material from the mouth of a dentistry patient.

### BACKGROUND OF THE INVENTION

Dental procedures often require a patient's mouth, or a portion of a patient's mouth to be kept relatively dry and clean of debris. Typically, while a dental practitioner performs a procedure, an assistant wields a saliva ejector device having a formable tube with one distal opening that receives saliva and other debris from the patient's mouth. Periodically, the dental practitioner pauses the procedure, and then the assistant passes the ejector device across excess material accumulating in and around the patient's mouth. In some cases, the distal opening of the ejector device may be formed into a hook-like shape and placed into the patient's mouth without the aid of an assistant, or the patient may be asked to close her lips around the ejector device, temporarily holding it in place.

The usual requirement that an assistant occasionally pass the device around the interior of the patient's mouth may be interruptive and inefficient for both the dental practitioner and the patient. Furthermore, the alternation between accumulation and removal of material may keep the patient's mouth insufficiently dry for a particular procedure.

Alternatively, portions of a patient's mouth may be kept dry by use of dams, absorbents, or other means. Such solutions may be obtrusive, bulky, uncomfortable, and inadequate.

### SUMMARY OF THE INVENTION

The present invention provides an apparatus adapted for improved ejection of material, e.g., saliva, from the mouth of a patient during a dental procedure, and related methods. The inventive apparatus may be alternatively referred to as a suction apparatus and saliva ejector. Generally, the inventive suction apparatus is designed to be connected to a suction/vacuum source and a waste collection container. The vacuum source provides the negative pressure to draw saliva and other debris from the oral cavity into the apparatus and eventually to the waste collection container.

In one aspect, the invention provides an apparatus comprising (i) a proximal tube having a first diameter, wherein the proximal tube is rigid, semi-rigid, or formable and adapted on the proximal end for connection to a suction and waste container apparatus, and (ii) two or more distal tubes each terminating in a porous extraction port and each having a second diameter smaller than the first diameter, wherein the distal tubes are flexible and in fluid communication with the proximal tube, wherein the porous extraction ports are sized for placement within an oral cavity of a subject.

In another aspect, the invention provides an apparatus comprising (i) a first tube and a second tube each having a proximal end and a distal end, wherein the tubes are adapted on the proximal end for connection to a suction and waste container apparatus, (ii) a first flexible tube and a second flexible tube in fluid communication with and attached to the distal end of the first tube and the second tube, respectively, and (iii) a first porous extraction port and a second porous extraction port distal to and in fluid communication with the first flexible tube and second flexible tube, respectively, wherein the porous extraction ports are sized for placement within an oral cavity of a subject. Optionally, the apparatus further comprises a first rigid or semi-rigid elbow tube disposed between the first flexible tube and the first extraction port, and a second rigid or semi-rigid elbow tube disposed between the second flexible tube and the second extraction port. The first tube and the second tube may be rigid, semi-rigid, or formable. Optionally, the first tube and the second tube each comprise a bend in the distal half of the tube and positioned in a manner that, in use, the bend secures the apparatus over the mandibular arch and the extraction ports are configured to be positioned in the lingual and/or buccal sulci.

In some embodiments of the foregoing aspects, the extraction ports comprise a central lumen in fluid communication with the distal tubes. In some embodiments, the extraction ports comprise a cotton roll. In some embodiments, the extraction ports are sized for placement within a buccal sulcus or a lingual sulcus.

In some embodiments, the apparatus comprises two, three, four, or more extraction ports. In other embodiments, the apparatus comprises two, three, four, or more distal tubes.

In another aspect, the invention provides a method for extracting saliva from a subject's mouth comprising:
(a) providing the apparatus of any one of the foregoing aspects;
(b) placing a first extraction port in a lingual sulcus or a buccal sulcus;
(c) placing a second extraction port in a lingual sulcus or a buccal sulcus; and
(d) applying a suction to the apparatus through the proximal tube, wherein the suction is sufficient to extract saliva from the subject's mouth through at least on of the extraction ports.

In some embodiments, the subject is a human or an animal. In other embodiments, the subject is a cat or a dog.

In some embodiments, one extraction port is placed in each lingual sulcus, one extraction port is placed in each buccal sulcus, or both. In another embodiment, one extraction port is placed in a buccal sulcus and another extraction port is placed in a lingual sulcus.

By “distal,” when referring to the inventive device, is meant toward the patient and away from the suction source. For example, the distal end(s) of the inventive device are the material extraction ports that are placed within the patient's mouth.

By “flexible,” when referring to a tube is meant one that has little to no linear rigidity and may be bent over a larger working range of angles than formable and semi-rigid tubes. Flexible tubes do not tend to return to a previous shape and do not readily maintain any particular shape and, therefore, do not add any significant structural support or rigidity to the device.

By “formable,” when referring to a tube including, for example, the proximal tube, is meant a tube that is semi-rigid such that the user may deform or otherwise shape the tube without damage to the tube wall or loss of patency and wherein the tube retains the formed shape. For example, standard saliva extractors known in the art are formable in that the suction tube is manufactured in a linear configuration but the user (e.g., dentist) may bend the extractor to facilitate saliva extraction in hard-to-reach areas of the oral cavity and/or to provide light retraction of soft tissues such as the cheeks and lips.

By “proximal,” when referring to the inventive device, is meant toward the suction source and away from the patient.

By “rigid,” when referring to a tube is meant one that cannot be significantly bent or otherwise deformed without a substantial risk of damage to the tube wall and/or loss of patency, and wherein the tube will not retain a deformed shape after the deformation pressure is released.

By “semi-rigid,” when referring to a tube is meant one that may be bent within a significant working range of angles without damage to the tube wall and/or loss of patency. Semi-rigid tubes may retain a deformed shape (i.e., also be “formable”) or may return or have bias to return to its original (e.g., manufactured) shape.

By “waste container apparatus” is meant a system which comprises a vacuum source (e.g., pump) and a waste collection container, as is typically used in dental and related procedures as described herein. Other tubing and fittings generally accompany the waste container apparatus in order to functionally connect that apparatus to the instrument (e.g., suction apparatus) used in the medical/dental procedure.

### DESCRIPTION OF THE DRAWINGS

The present invention may be better understood by referring to the following figures. The components in the figures are not necessarily to scale, emphasis instead being placed upon illustrating the principles of the disclosure. In the figures, reference numerals designate corresponding parts throughout the different views.

FIG. 1 is a schematic of a first implementation of a suction apparatus.

FIG. 2 is a perspective view of one exemplary placement of the material extraction ports on the buccal and lingual sides of a patient's mandibular arch.

FIG. 3 is a schematic of a second implementation of a suction apparatus.

FIG. 4A is a schematic side view illustrating one possible positioning of the suction apparatus in relation to the lower mandible of a subject. FIG. 4B is a schematic top view illustrating one possible positioning of the extraction ports relative to the lower mandible of a subject.

FIG. 5 is a schematic of a third implementation of a suction apparatus.

### DETAILED DESCRIPTION

The following description sets forth the invention and method of use in several embodiments. Those having ordinary skill in the art may be able to make alterations and modifications to what is described herein without departing from its spirit and scope. While this invention is susceptible to different embodiments in different forms, there is shown in the drawings and will herein be described in detail a preferred embodiment of the invention with the understanding that the present disclosure is to be considered as an exemplification of the principles of the invention and is not intended to limit the broad aspect of the invention to the embodiment illustrated. All features, elements, components, functions, and steps described with respect to any embodiment provided herein are intended to be freely combinable and substitutable with those from any other embodiment unless otherwise stated. Therefore, it should be understood that what is illustrated is set forth only for the purposes of example and should not be taken as a limitation on the scope of the present invention.

Saliva ejectors are commonly used by a dentist, dental hygienist, or other person of skill in the art who might require the removal of material such as saliva, blood, irrigation fluid (e.g., water), and other debris and fluid from a patient's oral cavity during a dental procedure. The present invention provides an improved saliva ejector having multiple extraction ports through which the unwanted/waste material (fluid) is removed by suction created by an external vacuum source (pump).

One purpose of the invention is to provide a saliva ejector having multiple extraction ports in order to more fully and efficiently remove unwanted fluids and debris from the oral cavity during a dental, medical, or surgical procedure. Generally, the ejector contains two extraction ports but it may be desirable to construct ejectors having three, four, or more extraction ports for use in certain procedures. Where the following descriptions refer to ejectors having two extraction ports, it is understood that additional extraction ports may be included for placement in other areas of the oral cavity. The extraction ports may be placed in any suitable location within the oral cavity and particularly in locations at which saliva and other fluids collect. For example, extraction ports may be placed into (i) the left and right lingual sulci, (ii) ipsilateral lingual sulcus and a buccal sulcus (e.g., the right lingual sulcus and right buccal sulcus or the left lingual sulcus and left buccal sulcus), (iii) contralateral lingual sulcus and a buccal sulcus (e.g., the right lingual sulcus and left buccal sulcus or the left lingual sulcus and right buccal sulcus), (iv) the left and right buccal sulci, and (v) higher order combinations thereof.

While in preferred embodiments, the suction apparatus is used to extract material from a human dental patient's oral cavity, the present invention is not necessarily be limited to application in human dentistry patients and could be used in non-human dental applications and/or non-dental medical applications. The sizing, configuration, and positioning of the various elements may be varied as appropriate for the desired use without departing from the spirit and scope of the invention.

Another purpose of the invention is to provide a saliva ejector that stays in place by itself without the need for to be manually held in place. It is common practice during dental, medical, and surgical procedures within the oral cavity to for the physician (e.g., surgeon or dentist) who is performing the procedure to be assisted by another individual (e.g., a dental hygienist, nurse, or other assistant), wherein the assistant is responsible for positioning and operating a saliva ejector. In many instances, the assistant is required to hold the saliva ejector for the entirety of the procedure, leaving the assistant unable or with reduced capacity to assist the physician with the procedure. Accordingly, it is desirable to have a saliva ejector that may be positioned in various locations within the oral cavity and that does not require constant attention by an operator. Such a device would allow a physician to perform certain procedures without an assistant and/or would free the assistant to perform other tasks required by the procedure.

In various aspects, the inventive saliva ejector generally comprises two or more extraction ports attached directly or indirectly to flexible tubing which facilitates comfortable and secure placement of the extraction ports at the target locations within the oral cavity. The flexible tubing attaches to formable tubing which is bendable by the operator but holds its shape under normal usage conditions. The formable tubing is connected directly or indirectly to a vacuum pump that provides the suction required to draw the fluids and debris from the oral cavity through the extraction ports, and to a collection vessel (waste container) that traps the unwanted fluid and debris for later disposal.

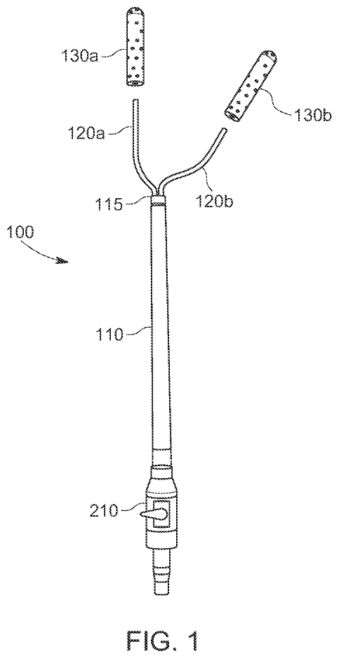

FIG. 1 provides an exploded view of one implementation of suction apparatus 100, shown ex situ. Suction apparatus 100 comprises a proximal tube 110 and a pair of flexible or semi-flexible distal tubes 120a, 120b attached to and in fluid communication with distal end of proximal tube 110. While FIG. 1 depicts two distal tubes 120a, 120b, in some embodiments, there may be 2, 3, 4, or more distal tubes. The distal ends of distal tubes 120a, 120b terminate in, and are in fluid communication with material extraction ports 130a, 130b. Extraction ports 130, distal tubes 120, proximal tube 110 are in fluid communication and the proximal end of the proximal tube 110 is operationally coupled to a waste container (not shown) and vacuum source (not shown) that generates a negative gas pressure sufficient to drawn waste fluids and debris through extraction ports 130. In some embodiments, the waste container and vacuum source have a standard configuration as is commonly found in dental offices. For example, optional adapter 210 is adapted to connect suction apparatus 100 to suction and waste collections systems standard and common in dental offices.

Optionally, distal tubes 120 may be connected to proximal tube 110 through end cap 115. In this embodiment, end cap forms a reversible or irreversible connection with proximal tube 110. Optionally, end cap 115 comprises a plurality of holes in a top surface equal to the number of distal tubes 120 connected to end cap 115, wherein distal tubes extend in the distal direction through the holes. Distal tubes 120 may have a flange, stopper, or other form of flared proximal end in order to secure distal tubes 120 to end cap 115. Many types of connections are known in the art and are suitable for connecting distal tubes 120 to proximal tube 110 provided that patency is maintained.

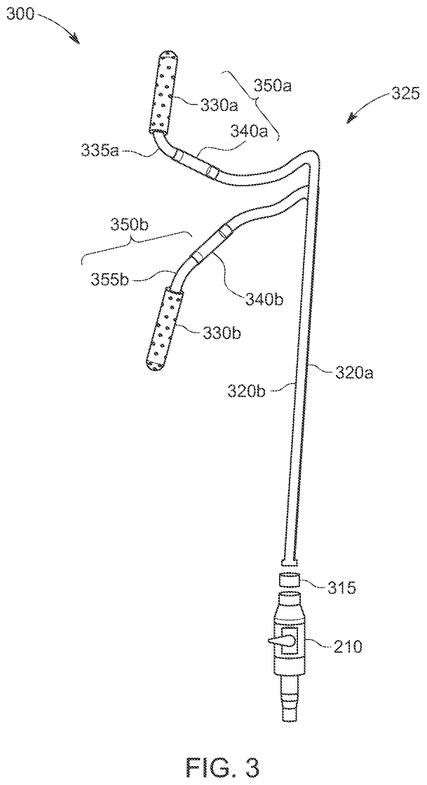

Proximal tube 110 and distal tubes 120 may be manufactured from any suitable material to any reasonable wall-thickness, diameter, and length. In some embodiment, the tubes are made from silicone, rubber, nylon, composite, polypropylene, PET, PTFE, and the like. In this implementation, distal tubes 120 are manufactured from a material and to specifications such that they may be easily and gently inserted into the patient's oral cavity, manipulated, positioned, arranged, adjusted, draped over the patient's mandibular arch, crimped, or removed. For example, FIG. 3 illustrates an exemplary placement of suction apparatus 100 along a patient's mandibular arch 200. Distal tubes 120a, 120b are freely moveable so that the material extraction ports 130a, 130b may be place in any desired location along the buccal sulci and/or lingual sulci. Suction apparatus 100 may be held in place simply by gravity and friction based on the positioning of the device on or against the patient. Optionally, an assistant may support suction apparatus 100 to ensure that material extraction ports 130 remain in the desired location within the oral cavity.

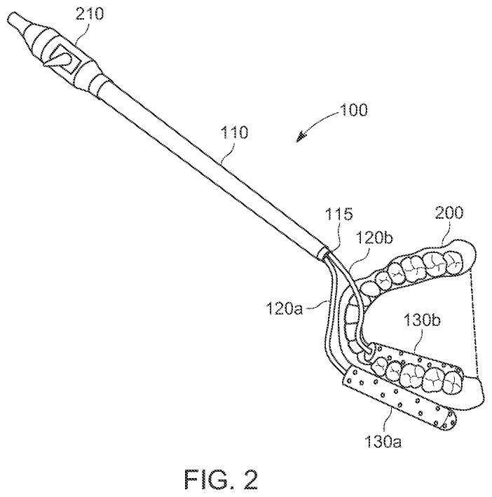

As shown in the exemplary placement in FIG. 2, the material extraction ports 130a, 130b are placed on the left buccal and left lingual sulci of the patient's mandibular arch 200. The suction apparatus 100 depicted in FIG. 2, has a proximal tube 110 fitted at its proximal end with an adapter 210 for fitting the suction apparatus 100 to standard dental suction units.

FIG. 3 provides a perspective view of another implementation of suction apparatus 300. As in the previous implementation, adapter 210 provides a connection between suction apparatus 300 and the vacuum source/waste container system (not shown) and, optionally, may provide a user-operated ball valve or similar device to regulate fluid flow and suction. Distal tubes 320a,b are in fluid communication with and connected on their proximal ends to adapter 210. This connection may be direct or indirect. FIG. 3 illustrates that distal tubes 320a,b are connected to adapter 210 through endcap 315. Alternatively or in addition to endcap 315, the connection may comprise a portion of proximal tube (not shown). The selection and design of the connection may be routinely varied based on the principles and requirements of design and end use.

In this implementation, distal tubes 320a,b are constructed from a rigid, semi-rigid, and/or formable material. A formable material is easily bendable by the user but retains its shape in use and under the weight of the device itself. The user is able to bend distal tubes 320a,b at bend 325 to bridge the teeth and/or mandibular arch 200 and maintain the desired placement of extraction ports 330a,b within the oral cavity. Distal tubes 320 may be manufactured to have a particular bend which may be unbendable or bendable by the user. Alternatively, Distal tubes 320 may be manufactured in a linear or other convenient configuration of a bendable material such that the user can customize the bend and fit for each patient.

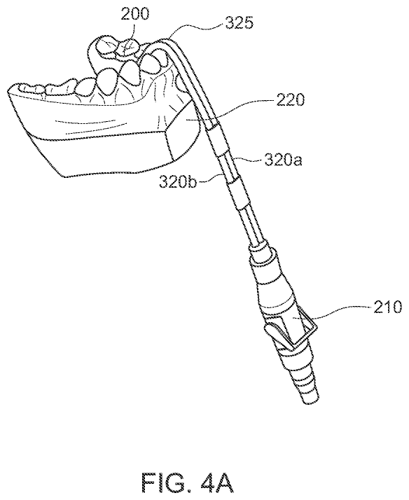

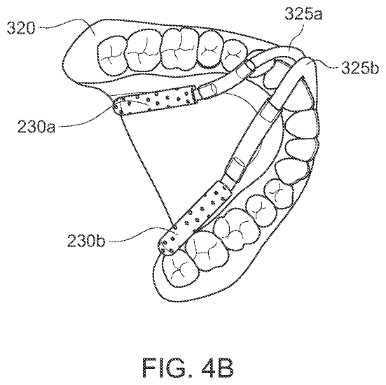

FIG. 4A shows a side view of suction apparatus 300 bridging the lower mandibular arch 200. Specifically, distal tubes 320a,b have bend 325 at a sufficient angle so as to maintain placement of extraction ports 330a,b (not shown) at the desired location. It is understood that bend 325 generally will have a less acute angle because dental patients typically are reclined in a supine position. FIG. 4B shows a top view of the lower mandible 220 for the positioning shown in FIG. 4A. Extraction ports 330a,b are shown as being positioned in the lingual sulci, however, one or both of extraction ports 330a,b could be positioned in the buccal sulci wherein neither or only one of bend 325a,b bridges the lower mandibular arch 200.

Distal tubes 320a,b are connected directly or indirectly to extraction ports 330a,b. Optionally, the connection is flexible in order to facilitate easy, secure, and comfortable placement of extraction ports 330a,b. In one embodiment, suction apparatus 300 optionally comprises flexible connector 350a,b that connects distal tubes 320a,b and extraction ports 330a,b, respectively. In one configuration, flexible connector 350 comprises, in proximal-to-distal order, flexible tube 340 and elbow 335 which connects distal tube 320 with extraction port 330. Elbow 335 may be rigid or semi-rigid, as necessary to facilitate placement and user comfort. In another embodiment, elbow 335 is omitted such that distal tube 320 is connected to extraction port 330 only through flexible tube 340.

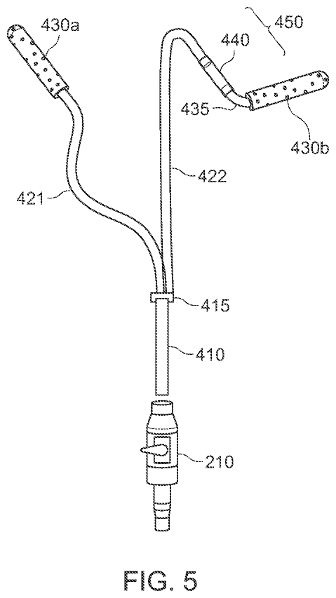

Although the inventive suction apparati are illustrated and described herein as being symmetrical, it is understood that the two side may be asymmetrical. For example, one side may have a long tube length than the other side to facilitate placement of extraction ports 330a,b in different bilateral locations. Furthermore, it is understood that the two sides need not have the same components and/or same arrangements. For example, FIG. 5 illustrates suction apparatus 400 in which one side has features of the first implementation and the other side has features of the second implementation. In this implementation, suction apparatus 400 comprises adapter 210, proximal tube 410, and endcap 415, as described above. First distal tube 421 is substantially the same as distal tubes 120 illustrated in FIG. 1 such that they are highly flexible and allow placement of extraction port 430a in any location within the oral cavity. Second distal tube 422 is substantially the same as distal tubes 320 illustrated in FIG. 3 and optionally may include flexible connector 450 comprising elbow 435 and/or flexible tube 440. In some embodiments, distal tube 422 is sufficiently rigid to support the weight of suction apparatus 400.

Proximal Tube and Adapter

A proximal tube as described herein provides a fluid connection at its proximal end with a suction source and contains and directs fluid and debris entering the suction apparatus through the extraction ports. The proximal tube may be substantially rigid, formable, semi-flexible, or flexible. It may comprise any standard high-volume evacuator (HVE), saliva ejector (SE), or aspirator tubing. The inner-bore diameter may range from ⅛″ up to 2″. The proximal tube may have any suitable wall thickness. The proximal tube may be smooth, corrugated, coiled, or any combination thereof, and may be substantially straight, curved, L-shaped, or in any other suitable linear configuration. The proximal tube may be made from extruded vinyl, silicone, polyvinylchloride (PVC), nylon, or other suitable tubing material. In some embodiments, the proximal tube is made from biodegradable materials such as plant-based polymers, paper, and bamboo.

The adapter 210 may be any suitable dental tubing coupler. For example, adapter 210 may be threaded or unthreaded; may have a “quick disconnect” barb insert fitting; and/or may have a flow-adjust and/or shutoff valve. The adapter may comprise PVC, nylon, steel, brass, aluminum, or other suitable material or combination of materials. Adapter 210 may be substantially straight, L-shaped (i.e., “elbow”), or otherwise angled. The adapter 210 may be rigid or may have a swivel joint or ball-and-socket joint.

Material Extraction Ports

Distal tubes are in fluid communication at their distal ends with the material extraction ports. The material extraction ports are sufficiently porous to permit saliva and other oral fluids and, optionally, small particulate debris to pass from the oral cavity into the distal tubes. In some embodiments, the porosity of the extraction ports is small enough such that oral tissues (e.g., tongue, cheek, lips, etc.) are not trapped by suction, thereby causing discomfort to the patient and impeding the extraction of saliva. Extraction ports may comprise any convenient shape and suitable porous material. In preferred embodiments, material extraction ports 130 are substantially cylindrical and have a greater diameter than that of the distal tubes. In some embodiments, extraction ports are manufactured from cotton rolls having a central bore in fluid communication with the distal ends of distal tubes.

Material extraction ports may be of substantially uniform composition, or may optionally be perforated, i.e., disposed with a series of holes along the surface to permit increased flow of gases and fluids. Thus, material, e.g., saliva, flows through the nap and/or surface holes and into the distal tubes, down into the proximal tube, and then may be collected or discarded as waste. Alternatively, the material extraction ports may have 1, 2, 3, 4, or more additional holes of any suitable size or location on the surface for receiving material from a patient's oral cavity.

In some embodiments, the material extraction ports comprise a solid cap fitting over the distal end of the distal tubes the caps having a variously-shaped openings, e.g., circular, cross-shaped, star-shaped, polygonal, etc., over the opening to the lumens of the distal tubes.

The invention has been described broadly and generically herein. Each of the narrower species and subgeneric groupings falling within the generic disclosure also form part of the methods. This includes the generic description of the methods with a proviso or negative limitation removing any subject matter from the genus, regardless of whether or not the excised material is specifically recited herein.

Other embodiments are within the following claims. In addition, where features or aspects of the methods are described in terms of Markush groups, those skilled in the art will recognize that the invention is also thereby described in terms of any individual member or subgroup of members of the Markush group.

1. An apparatus comprising:
a proximal tube having a first diameter, wherein the proximal tube is rigid, semi-rigid, or formable and adapted on the proximal end for connection to a suction and waste container apparatus;
two or more distal tubes each terminating in a porous extraction port and each having a second diameter smaller than the first diameter, wherein the distal tubes are flexible and in fluid communication with the proximal tube; and
wherein the porous extraction ports are sized for placement within an oral cavity of a subject.

2. The apparatus of claim 1, wherein the apparatus further comprises waste container apparatus comprising a suction source and a waste container.

3. The apparatus of claim 1, wherein the extraction ports comprise a central lumen in fluid communication with the distal tubes.

4. The apparatus of claim 1, wherein the extraction ports comprise a cotton roll.

5. The apparatus of claim 1, wherein the apparatus has two distal tubes.

6. The apparatus of claim 1, wherein the extraction ports are sized for placement within a buccal sulcus or a lingual sulcus.

7. The apparatus of claim 1, wherein the subject is a human.

8. The apparatus of claim 1, wherein the subject is an animal.

9. The apparatus of claim 8, wherein the animal is a dog or a cat.

10. An apparatus comprising:
a first tube and a second tube each having a proximal end and a distal end, wherein the tubes are adapted on the proximal end for connection to a suction and waste container apparatus;
a first flexible tube and a second flexible tube in fluid communication with and attached to the distal end of the first tube and the second tube, respectively;
a first porous extraction port and a second porous extraction port distal to and in fluid communication with the first flexible tube and second flexible tube, respectively, wherein the porous extraction ports are sized for placement within an oral cavity of a subject.

11. The apparatus of claim 10, wherein the apparatus further comprises a first rigid or semi-rigid elbow tube disposed between the first flexible tube and the first extraction port, and a second rigid or semi-rigid elbow tube disposed between the second flexible tube and the second extraction port.

12. The apparatus of claim 10, wherein the first tube and the second tube are rigid, semi-rigid, or formable.

13. The apparatus of claim 10, wherein the first tube and the second tube each comprise a bend in the distal half of the tube.

14. The apparatus of claim 10, wherein the apparatus further comprises waste container apparatus comprising a suction source and a waste container.

15. The apparatus of claim 10, wherein the extraction ports comprise a central lumen in fluid communication with the first and second tubes.

16. The apparatus of claim 10, wherein the extraction ports comprise a cotton roll.

17. The apparatus of claim 10, wherein the extraction ports are sized for placement within a buccal sulcus or a lingual sulcus.

18. The apparatus of claim 10, wherein the subject is a human.

19. The apparatus of claim 10, wherein the subject is an animal.

20. The apparatus of claim 19, wherein the animal is a dog or a cat.

21.-24. (canceled)
